(19) United States
(12) Patent Application Publication
(10) Pub. No.: US 2002/0055654 A1
(43) Pub. Date: May 9, 2002

(54) Method for producing benzyl derivitives of 2-butene-1,4 diols

(76) Inventors: Qi Wang, Reading, PA (US); Ramesh Krishnamurti, Bangalore (IN); Thomas F. Smolka, West Seneca, NY (US)

Correspondence Address: Joseph D. Yao, Occidental Chemical Corporation, 5005 LBJ Freeway, Dallas, TX, 75244, US

(21) Appl. No.: 09/950,181

(22) Filed: Sep. 10, 2001

Related U.S. Application Data
(60) Provisional application No. 60/231,790, filed on Sep. 11, 2000.

(51) Int. Cl. C07C 319/14; C07C 41/01

(52) U.S. Cl. 568/39; 568/662

(57) ABSTRACT

A method for producing benzyl derivatives of 2-butene-1,4-diols by reacting 2-butene-1,4-diols with benzyl chloride compounds in the presence of an alkali or alkaline earth metal hydroxide or carbonate to produce benzyl derivatives of 2-butene-1,4-diols and recovering at least a portion of at least one such benzene derivative.

### RELATED APPLICATIONS

This application is entitled to and hereby claims the filing date of provisional application No. 60/231,790, “ALKYLATION OF 2-BUTENE-1,4-DIOL” Filed Sep. 11, 2000 by Qi Wang, Ramesh Krishnamurti and Thomas F. Smolka.

### FIELD OF THE INVENTION

Benzyl derivatives of 2-butene-1,4-diols, such as these of parent 2-butene-1,4-diol are efficient stabilizers which protect polymers from discoloration in the presence of gamma radiation. The mono-benzylated derivatives are particularly effective to protect polymers against discoloration. This invention deals with methods for producing benzyl derivatives of 2-butene-1,4-diols.

### BACKGROUND OF THE INVENTION

Benzyl derivatives of 2-butene-1,4-diol, such as 4-benzyloxy-2-butene-1-ol, and 1,4-dibenzyloxy-2-butene are very efficient stabilizers that protect polymers from discoloration in the presence of gamma radiation. The mono-benzylated derivatives are preferred, but both the di and mono-benzylated derivatives are effective.

Mono-benzylated derivatives such as 4-benzyloxy-2-butene-1-ol have previously been prepared by two methods. The first is a two-stage method involving their benzylideneacetals as intermediates followed by reduction with reducing agents such as lithium aluminum hydride. The second uses expensive benzyl bromide as an alkylating agent. These processes are disclosed in “A Stereospecific Route To Aziridinomitosanes: The Synthesis of Novel Mitomycin Congeners,” S. Danishefsky, E. M. Berman, M. Ciufolini, S. J. Etheredge, B. F. Segmuller, J.Am. Chem. Soc, 1985, V.107, 3891-3898); and “Convenient One-pot Synthesis of (Z)-4-Benzyloxy-2-buten-1-ol,” S. V. Hiremath, D. R. Reddy, M. A. Kumar, Indian J. Chem, 1988, V.27B, 558.

Neither of these techniques is cost-effective to produce the desired benzyl derivatives of 2-butene-1,4-diol for widespread use to prevent discoloration of polymers.

Accordingly, since these benzyl derivatives are known to be extremely effective for preventing yellowing, a continuing effort has been directed to the development of a process for producing these materials more economically.

### SUMMARY OF THE INVENTION

The present invention comprises a method for producing benzyl derivatives of 2-butene-1,4-diols, the method comprising:

a) reacting in a reaction zone

1) a butenediol compound having the general formula

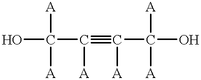

wherein the diol is in either in cis or trans form. A is independently selected from hydrogen, halogen, R, OR, and SR, wherein R is an alkyl group containing from 1 to about 12 carbon atoms, or an alkylaryl, an arylalkyl group or an aryl group containing from 6 to about 12 carbon atoms; and

2) a benzyl chloride compound having the general formula formula

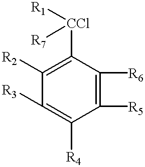

wherein R₁, R₂, R₃, R₄, R₅, R₆ and R₇ are independently selected from hydrogen, R, OR, SR and RCO and R is an alkyl group containing from 1 to about 12 carbon atoms, an alkylaryl, an arylalkyl, or an aryl group containing from 6 to about 12 carbon atoms; in the presence of an alkali or an alkaline earth metal hydroxide or carbonate to produce at least one benzyl derivative of the butenediol compound as a product; and,

b) recovering at least a portion of the at least one benzyl derivative.

The invention further comprises a method for producing benzyl derivatives of the parent 2-butene-1,4-diol, the method comprising:

a) reacting in a reaction zone 2-butene-1,4-diol with a benzyl chloride compound having the general formula

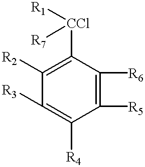

wherein R₁, R₂, R₃, R₄, R₅ R₆ and R₇ are selected from hydrogen, R, OR, SR and RCO and R is an alkyl group containing from 1 to about 12 carbon atoms, an alkylaryl, arylalkyl, or an aryl group containing from 6 to about 12 carbon atoms in the presence of an alkali or an alkaline earth metal hydroxide or carbonate to produce at least one benzyl derivative of 2-butene-1,4-diol; and,

b) recovering at least a portion of the at least one benzyl derivative of 2-butene-1,4-diol.

The method further comprises a method for producing benzyl derivatives of 2-butene-1,4-diol, the method consisting essentially of reacting 2-butene-1,4-diol with benzyl chloride having the formula

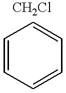

in a reaction zone in the presence of an alkali or alkaline earth metal hydroxide or carbonate selected from the group consisting of sodium hydroxide, sodium carbonate, potassium hydroxide and potassium carbonate to produce at least one benzyl derivative of 2-butene-1,4-diol and recovering at least a portion of the at least one benzyl derivative.

### DESCRIPTION OF THE PREFERRED EMBODIMENTS

According to the present invention, it has been found that benzyl chlorides, can be used to prepare the desired benzyl derivatives of 2-butene-1,4-diols. The benzyl chlorides are readily produced industrially by a process which in general comprises bubbling through, or otherwise contacting, a chlorine-containing gas with toluenes, preferably in the presence of ultra-violet light. Benzyl chlorides are considered to be readily available commercially and no further discussion of the preparation of these materials is considered necessary.

The benzyl chlorides can take a wide range of forms, of the general formula

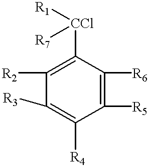

wherein R₁, R₂, R₃, R₄, R₅ R₆ and R₇ are independently selected from hydrogen, R, OR, SR and RCO and R is an alkyl group containing from 1 to about 12 carbon atoms, an alkylaryl, arylalkyl, or an aryl group containing from about 6 to about 12 carbon atoms. Preferably, R₁ and R₇ are hydrogen atoms and the benzyl chloride has the following formula.

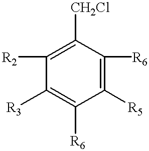

It is also preferred that the remaining R groups, i.e., R₂, R₃, R₄, R₅ and R₆, are hydrogen. This is the simplest benzyl chloride structure, is commonly referred to as benzyl chloride and is the most readily available. While the more complex structures can be used, it is believed that the benzyl chloride described above will be found to be the most economical and the most readily available.

In the preparation of the desired benzyl derivatives of 2-butene-1,4-diols, the preferred reactant is the parent 2-butene-1,4-diol. Other materials could be used as represented by the general formula

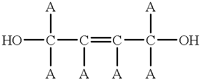

wherein the diol is in either cis or trans form. A is independently selected from hydrogen, halogen, R, OR, and SR, wherein R is an alkyl group containing from 1 to about 12 carbon atoms, or an alkylaryl, an arylalkyl group or an aryl group containing from 6 to about 12 carbon atoms.

Preferably, A is hydrogen in all instances. The instance where A is hydrogen represents the parent 2-butene-1,4-diol which is preferred both because it is readily available commercially and because it readily reacts with the benzyl chloride in the process of the present invention. It is preferred that the reactants are benzyl chloride and 2-butene-1,4-diol. These materials are desirably reacted in the presence of a base such as an alkali or alkaline earth metal hydroxide or carbonate to produce at least one benzyl derivative. Preferred alkali or alkaline earth metal hydroxides or carbonates are sodium hydroxide, potassium hydroxide, sodium carbonate and potassium carbonate.

In the reaction of these materials, it may be desirable in some instances to dissolve the alkali or alkaline earth material in the 2-butene-1,4-diol prior to mixing the benzyl chloride compound with the diol compound. In such instances, the reaction can be conducted with only the reagents present. It is preferable that the reaction temperature is from about 50 to about 120° C. Desirably, the reaction is conducted at a temperature from about 80 to about 120° C. In the reaction zone, it is desirable that the reactants are present in a molar ratio 2-butene-1,4-diol: benzyl chloride compound: base of 1:0.1:0.1 to 1:16:16. Suitable reaction times are from about 0.2 to about 90 hours at the reaction temperature above.

In some instances, it may be desirable to add a phase transfer catalyst to the reaction zone, especially when the amount of bis-benzylated derivative needs to be increased in the final product. One such suitable catalyst is benzyltriethylammonium chloride. Any commonly used phase transfer catalyst which is effective with the reactants can be used for this purpose. They include various phase transfer catalysts, such as organic quaternary ammonium salts, organic guanidinium salts, organic phosphonium salts, crown ethers, and various polyethers such as poly(ethylene glycol) and poly(propylene oxide).

If desired, a solvent can be used in the reaction zone. The solvent can be water and organic solvents such as N,N-dimethylformamide, dimethyl sulfoxide, dimethyl sulfone, sulfolane, pyridine, piperidine, t-butyl methyl ether, paraffinic solvents, and toluene. Desirably the selected solvent has a boiling point above 50° C. at atmospheric pressure.

In some instances, it is desirable to prepare the bis-benzyl derivatives of 2-butene-1,4-diol. These materials are commonly produced in combination with the mono benzyl compounds and can be used in combination if desired. The production of the bis-benzyl derivatives is facilitated by the use of the phase transfer catalyst.

The process of benzylating 2-butene-1,4 diol is illustrated with following scheme. In the process, it is desirable to adjust the starting materials ratios according to the relative amount of mono and bis benzylated products needed in the formulated polymer stabilizer. The excess of starting materials can be readily recovered and utilized for the production of additional products. In the case of using excessive 2-butene-1,4-diol, the unreacted diol can be readily separated from products by draining out the bottom phase of the butenediol. If excess of benzyl chloride is used, simple distillation can be used to recover the starting material.

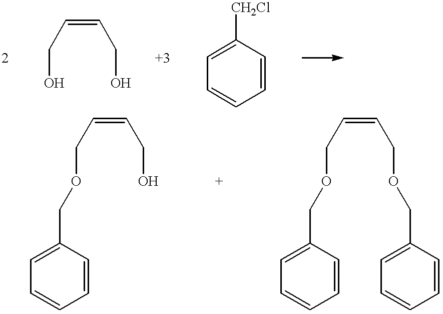

The remaining alkali or alkaline earth metal hydroxide or carbonate and the reaction side product alkali metal or alkaline earth metal chlorides are readily removed by washing the final reaction mixture with water. The resulting benzyl derivatives of 2-butene-1,4-diol are then usable after drying as stabilizers for polymers. If higher purity is specified for the benzylation products, distillation can used for the further purification.

Polymers, which may be protected using these materials, are poly(vinyl chloride), poly(vinylidene chloride), polyolefines such as polyethylene and polypropylene, polyacrylates, polycarbonates, polyesters, polyamides, and polyimides.

When a solvent is used, desirably molar ratio of 2-butene-1,4-diol: benzyl chloride compound: alkali or alkaline earth metal hydroxide or carbonate: solvent can vary from about 1:0.1:0.1:0.1 to about 1:16:16:100, preferably, this ratio is from about 1:0.8:0.8:2 to about 1:2:2:10. In the absence of solvent, desirably the molar ratio of 2-butene-1,4-diol: benzyl chloride compound: alkali or alkaline earth metal hydroxide or carbonate can vary from about 1:0.1:0.1 to about 1:16:16, preferably, this ratio is from about 1:0.5:0.5 to about 1:2:2.

### EXAMPLE I

A 500 milliliter three-necked flask equipped with a thermometer, thermo-watch, reflux condenser and a mechanical stirrer were used for the experiment. In the experiment, 0.6 moles of 2-butene-1,4-diol and 0.6 moles of sodium hydroxide were charged to the flask. The mixture was then heated to 120° C. for twenty minutes to dissolve the sodium hydroxide. The solution was then cooled down to 70° C. and 0.6 moles of benzyl chloride was added drop-wise to the flask. After the addition, the reaction temperature was raised and controlled at about 120° C. for one hour. The reaction mixture was then cooled and washed with brine and water. The reaction mixture was then dried over magnesium sulfate.

Additional comparable experiments were run as shown below in Table I. In all cases, the diol used is 2-butene-1,4-diol. The chloride in all instances is benzyl chloride. The base is shown and the solvent was all toluene.

The reaction temperatures, times and reaction products are shown in the Table.

TABLE I

| | | | | | 4- | | |
|---|---|---|---|---|---|---|---|
| | | | | Reaction | benzyloy | Dibenzyloxy | Phase |
| | Molar | Base-Tests | Reaction | Time | -2-buten- | 2-butene | Transfer |
| Test | Ratio* | 1-7 | Temp (° C.) | (total hr) | 1-ol(%) | (%) | Catalyst |
| 1 | 1:1:1:0 | Solid NaOH | 120 | 1 | 63.6 | 26.0 | none |
| 2 | 1:1:1:0 | Solid NaOH | 120 | 2 | 58.9 | 31.3 | none |
| 3 | 1.0:17:0.70 | Solid NaOH | 120 | 4 | 86.6 | 8.4 | none |
| 4 | 1:0.5:0.5:0 | Solid NaOH | 120 | 4 | 77.1 | 14.8 | none |
| 5 | 10:33:0.33:0 | Solid NaOH | 120 | 0.2 | 85.8 | 10.5 | none |
| 6 | 1:1:1:3.1 | Solid NaOH | 120 | 2.5 | 71.2 | 16.9 | none |
| 7 | 1:1:1:3.1 | Solid NaOH | 120 | 12 | 71.8 | 14.9 | none |
| 8 | 1:1:1:3.1 | 50% aqueous | 100 | 3.2 | 75.7 | 10.4 | none |
| | | NaOH | | | | | |
| 9 | 1:1:1:3.1 | 50% aqueous | 90 | 3 | 72.7 | 3.6 | none |
| | | NaOH | | | | | |
| 10 | 1:1:1.2:3.1 | 50% aqueous | 90 | 4.5 | 70.9 | 5.4 | none |
| | | NaOH | | | | | |
| 11 | 1:1:1.2:3.1 | 50% aqueous | 90 | 4.5 | 73.7 | 4.6 | DowFax |
| | | NaOH | | | | | hydrotrope |
| 12 | 1:4.3.15.5:0 | 50% aqueous | 80 | 3.0 | 0 | 100 | Benzyltriet |
| | | NaOH | | | | | hylam- |
| | | | | | | | monium |
| | | | | | | | chloride |

*The diol: the choride: the base: the solvent
Yield is based on the limiting reagent.

When aqueous sodium hydroxide is used, it can be mixed directly with the butene diol.

In test 12, a benzyl triethylammonium chloride was used as a phase transfer catalyst. With this catalyst, substantially all of the product was the di-benzyl derivative.

It is also noted, that in all the cases, high yields of the benzylated butenediol were obtained.

In the table, the yields are based upon the percentage conversion of the stoichometrically limiting reagent.

In view of the foregoing disclosure, it is believed clear that benzyl derivatives of 2-butene-1,4-diol are readily produced by the process of the present invention.

Having thus described the invention by reference to certain of its preferred embodiments, it is respectfully pointed out that the embodiments described are illustrative rather than limiting in nature and that many variations and modifications are possible within the scope of the present invention. Having thus described the invention, we claim:

1. A method for producing benzyl derivatives of 2-butene-1,4-diols, the method comprising:
a) reacting in a reaction zone
1) a butene diol compound having the general formula 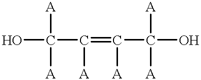
wherein the diol is in either in cis or trans form. A is independently selected from hydrogen, halogen, R, OR, and SR, wherein R is an alkyl group containing from 1 to about 12 carbon atoms, or an alkylaryl, an arylalkyl group or an aryl group containing from 6 to about 12 carbon atoms; and
2) a benzyl chloride compound having the general formula formula 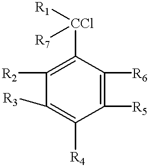
wherein R₁, R₂, R₃, R₄, R₅, R₆ and R₇ are independently selected from hydrogen, R, OR, SR and RCO and R is an alkyl group containing from 1 to about 12 carbon atoms, an alkylaryl, an arylalkyl, or an aryl group containing from 6 to about 12 carbon atoms; in the presence of an alkali or an alkaline earth metal hydroxide or carbonate to produce at least one benzyl derivative of the butenediol compound as a product; and,
b) recovering at least a portion of the at least one benzyl derivative.

2. The method of claim 1 wherein R₁ and R₇ are hydrogen.

3. The method of claim 2 wherein R₂, R₃, R₄, R₅ and R₆ are hydrogen.

4. The method of claim 1 wherein the benzyl chloride compound is the parent benzyl chloride.

5. The method of claim 1 wherein A is hydrogen.

6. The method of claim 1 wherein the butene diol compound is 2-butene-1,4-diol.

7. The method of claim 1 wherein the benzyl derivative of the butene diols product is at least one of a mono-benzylated and a bis-benzylated derivative of the butene diol compound.

8. A method for producing benzyl derivatives of 2-butene-1,4-diol, the method comprising:
a) reacting in a reaction zone 2-butene-1,4-diol with a benzyl chloride compound having the general formula 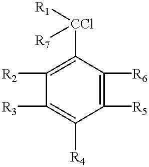
wherein R₁, R₂, R₃, R₄, R₅ R₆ and R₇ are selected from hydrogen, R, OR, SR and RCO and R is an alkyl group containing from 1 to about 12 carbon atoms, an alkylaryl, arylalkyl, or an aryl group containing from 6 to about 12 carbon atoms in the presence of an alkali or an alkaline earth metal hydroxide or carbonate to produce at least one benzyl derivative of 2-butene-1,4-diol; and,
b) recovering at least a portion of the at least one benzene derivative of a 2-butene-1,4-diol.

9. The method of claim 8 wherein R₁ and R₇ are hydrogen.

10. The method of claim 8 wherein R₂, R₃, R₄, R₅ and R₆ are hydrogen.

11. The method of claim 8 wherein R₁ and R₇ are hydrogen and wherein R₂, R₃, R₄, R₅ and R₆ are hydrogen.

12. The method of claim 8 wherein the alkali or alkaline earth metal hydroxide or carbonate is selected from the group consisting of sodium hydroxide, potassium hydroxide, sodium carbonate and potassium hydroxide.

13. The method of claim 8 wherein the alkali or alkaline earth hydroxide or carbonate is sodium hydroxide.

14. The method of claim 13 wherein the 2-butene-1,4-diol: benzyl chloride compound: alkali or alkaline earth metal hydroxide or carbonate are present in a molar ratio from 1:0.1:0.1 to 1:16:16.

15. The method of claim 8 wherein a solvent is added to the reaction zone.

16. The method of claim 15 wherein the solvent is selected from the group consisting of water and organic solvents such as N,N-dimethylformamide, dimethyl sulfoxide, dimethyl sulfone, sulfolane, pyridine, piperidine, t-butyl methyl ether, paraffinic solvents and toluene having boiling points above 50° C. at atmospheric pressure.

17. The method of claim 8 wherein the reaction temperature is from about 50 to about 120° C.

18. A method for producing benzyl derivatives of a 2-butene-1,4-diol, the method consisting essentially of reacting 2-butene-1,4-diol with benzyl chloride in a reaction zone in the presence of an alkali or alkaline earth metal hydroxide or carbonate selected from the group consisting of sodium hydroxide, sodium carbonate, potassium hydroxide and potassium carbonate to produce at least one benzyl derivative of 2-butene-1,4-diol and recovering at least a portion of the at least one benzyl derivative.

19. The method of claim 18 wherein the 2-butene-1,4-diol and the benzyl chloride are reacted at a temperature from about 50 to about 120° C. for a time from about 0.2 to about 90 hours.

20. The method of claim 18 wherein the at least one benzyl derivative comprises at least one of 4-benzyloxy 2-butene-1-ol and di-benzyloxy-2-butene.

21. The method of claim 18 wherein a catalyst is added to the reaction zone.

22. The method of claim 21 wherein the catalyst is benzyl triethylammonium chloride.

23. The method of claim 18 wherein the alkali or alkaline earth metal hydroxide or carbonate is at least partially dissolved in the 2-butene-1,4-diol prior to introduction of the benzyl chloride into the reaction zone.

24. The method of claim 18 wherein the alkali or alkaline earth metal oxide or carbonate is sodium hydroxide.

25. The method of claim 24 wherein a solvent is present in the reaction zone.

26. The method of claim 25 wherein the solvent is water and organic solvents such as N,N-dimethylformamide, dimethyl sulfoxide, dimethyl sulfone, sulfolane, pyridine, piperidine, t-butyl methyl ether, paraffinic solvents and toluene having boiling points above 50° C. at atmospheric pressure.

27. The method of claim 26 wherein the sodium hydroxide is present in the reaction zone as an aqueous solution of sodium hydroxide.

28. The method of claim 27 wherein the benzyl chloride is added to the aqueous solution of sodium hydroxide.
